(19) United States
(12) Patent Application Publication
(10) Pub. No.: US 2008/0275308 A1
(43) Pub. Date: Nov. 6, 2008

(54) Premium-Based Civilly-Catastrophic Event Threat Assessment

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/539,861

(22) Filed: Oct. 9, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.
Application No. 11/465,063 is a continuation-in-part of application No. 11/466,727, filed on Aug. 23, 2006.
Application No. 11/466,727 is a continuation-in-part of application No. 11/466,953, filed on Aug. 24, 2006.
Application No. 11/466,953 is a continuation-in-part of application No. 11/470,156, filed on Sep. 5, 2006.
Application No. 11/470,156 is a continuation-in-part of application No. 11/531,651, filed on Sep. 13, 2006.
Application No. 11/531,651 is a continuation-in-part of application No. 11/532,461, filed on Sep. 15, 2006.
Application No. 11/532,461 is a continuation-in-part of application No. 11/535,021, filed on Sep. 25, 2006.
Application No. 11/535,021 is a continuation-in-part of application No. 11/535,282, filed on Sep. 26, 2006.
Application No. 11/535,282 is a continuation-in-part of application No. 11/537,469, filed on Sep. 29, 2006.
Application No. 11/537,469 is a continuation-in-part of application No. 11/539,798, filed on Oct. 9, 2006.

(51) Int. Cl. A61B 5/00

(52) U.S. Cl. 600/300

(57) ABSTRACT

One accepts (101) consideration-based private civil security subscriptions with respect to providing the civilly-catastrophic event threat assessment. One gathers (102) information regarding specific civilly-catastrophic event causation agents. One may optionally gather (103) information regarding an authorized beneficiary. One then uses (104) the gathered information, at least in part, to formulate a civilly-catastrophic event threat assessment. One then provides (105) a civilly-catastrophic event threat assessment. One may optionally provide (106) the authorized beneficiary with communication equipment. One may optionally provide (107) survival instructions to the authorized beneficiary. One may optionally prompt (108) anticipatory steps by the authorized beneficiary. One may optionally provide (109) access to at least one life-sustaining resource. One may optionally provide (110) corresponding services and/or information.

### RELATED APPLICATIONS

This comprises a continuation-in-part of each of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265;

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043;

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605;

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application Ser. No. 11/464,751;

PRIVATELY PROVISIONED SURVIVAL SUPPLIES SUB UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

WASTE DISPOSAL DEVICE as filed on Aug. 16, 2006 and having application Ser. No. 11/465,063;

SUBSCRIPTION-BASED PRIVATE CIVIL SECURITY RESOURCE CUSTOMIZATION METHOD as filed on Aug. 23, 2006 and having application Ser. No. 11/466,727;

PREMIUM BASED PRIVATE CIVIL SECURITY POLICY METHODS as filed on Aug. 24, 2006 and having application Ser. No. 11/466,953;

SUBSCRIPTION-BASED MOBILE SHELTER METHOD as filed on Sep. 5, 2006 and having application Ser. No. 11/470,156;

METHOD OF PROVIDING A FLOATING LIFE-SUSTAINING FACILITY as filed on Sep. 13, 2006 and having application Ser. No. 11/531,651;

PRIVATELY PROVISIONED SUB-UNIT-BASED SURVIVAL SUPPLIES PROVISIONING METHOD as filed on Sep. 15, 2006 and having application Ser. No. 11/532,461;

PRIVATELY PROVISIONED INTERLOCKING SUB UNIT BASED SURVIVAL SUPPLIES PROVISIONING METHOD as filed on Sep. 25, 2006 and having application Ser. No. 11/535,021;

RESOURCE CONTAINER AND POSITIONING METHOD AND APPARATUS as filed on Sep. 26, 2006 and having application Ser. No. 11/535,282;

PUBLICLY-FUNDED PRIVATELY FACILITATED ACCESS TO SURVIVAL RESOURCES METHOD as filed on Sep. 29, 2006 and having application Ser. No. 11/537,469;

ELECTRICITY PROVIDING PRIVATELY PROVISIONED SUBSCRIPTION BASED SURVIVAL SUPPLY UNIT METHOD AND APPARATUS as filed on Oct. 9, 2006 and having application Ser. No. 11/539,798;

the contents of each of which are fully incorporated herein by this reference.

### TECHNICAL FIELD

This invention relates generally to providing civilly-catastrophic event threat assessments.

### BACKGROUND

Many citizens of the world have long passed the point when a ready availability of the basic necessities of life is satisfactory in and of itself. Today's consumer-oriented citizens demand, and often receive, an incredibly diverse and seemingly ever growing cornucopia of consuming and experiential options. Such riches are typically based, in turn, upon a highly interdependent series of foundational infrastructure elements. Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related actions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swath of a given civil entity. Any number of natural and/or non-natural events can greatly disrupt society's infrastructure and corresponding ability to provide one or more life-sustaining resources such as water, nutrition, shelter, and the like.

Many people believe and trust that their government (local, regional, and/or national) will reliably assist them with respect to predicting civilly-catastrophic events and provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to reliably forecast the impact of a given civilly-catastrophic event or to respond, before or after the fact, to even basic survival needs when a civilly-catastrophic event occurs. To a large extent one may reasonably argue that many modern governments have forsaken their responsibility to design, fund, implement, or even discuss an effective civil defense program capable of protecting large segments of their populations.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum. On the one hand, modern governments typically do little to proactively ensure the survival (let alone the comfort) of their citizens in the face of most civilly-catastrophic events. Governmental authorities often provide insufficient (or late) information regarding civil threats of various kinds. Concerned individuals often find themselves with insufficient information regarding specific threats in this regard, including the existence of the threat, the characterizing nature of the threat, meaningful actions that one can take to better ensure one's own survival in the face of the threat, and so forth.

On the other hand, attempting to take responsible actions to independently obtain such information can become, in and of itself, nearly a full-time avocation and leave little time to actually enjoy the conveniences and opportunities of modern life. Such individual actions may even be frowned upon by the greater part of society which has grown accustomed and falsely secure with existing efficient just-in-time delivery systems that provide the illusion of plenty while undercutting the perception of risk.

### BRIEF DESCRIPTION OF THE DRAWING

The above needs are at least partially met through provision of the premium-based civilly-catastrophic event threat assessments described in the following detailed description, particularly when studied in conjunction with the drawing, wherein:

FIG. 1 comprises a flow diagram as configured in accordance with various embodiments of the invention.

Skilled artisans will appreciate that elements in the figure are illustrated for simplicity and clarity. For example, common but well-understood elements that are useful or necessary in a commercially-feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, one accepts consideration-based private civil security subscriptions from subscribers with respect to providing a civilly-catastrophic event threat assessment to an authorized beneficiary. One gathers information regarding specific civilly-catastrophic event causation agents and the information is used, at least in part, to formulate a civilly-catastrophic event threat assessment. One then provides the civilly-catastrophic event threat assessment to the authorized beneficiary. So configured, the authorized beneficiary is able to reliably and predictably receive specific information regarding potential civilly-catastrophic events.

So configured, the civilly-catastrophic threat assessment information can be both highly personal and customized to reflect the needs of a given authorized beneficiary. Authorized beneficiaries will be able to become generally better informed regarding various civilly-catastrophic threats and to be generally better prepared to more predictably and reliably survive a variety of such civilly-catastrophic events. In addition, authorized beneficiaries as noted above will be able to reliably and predictably receive specific information regarding recommended actions as correspond to their consideration-based private civil security subscriptions. Accordingly, the authorized beneficiaries can take important steps to bring a considerably improved measure of security into their lives without having to effectively become a full-time survivalist; such individuals can, in short, continue to enjoy their chosen vocations and standard of living knowing that, should a civilly-catastrophic event be predicted, they will have extraordinary access to threat assessments and informational resources that will greatly enhance their survival opportunities should the civilly-catastrophic event occur.

The gathering of information regarding civilly-catastrophic threat assessment causation agents, using the information to formulate civilly-catastrophic threat assessments, and provision of civilly-catastrophic event threat assessments to an authorized beneficiary are readily facilitated without dependency upon governmental oversight, participation, or control (though in some embodiments it may be desired, for example, to build relationships with government entities in order to facilitate the exchange of intelligence related to civilly-catastrophic event causation agents, of information pertaining to post-event responses and plans, and so forth).

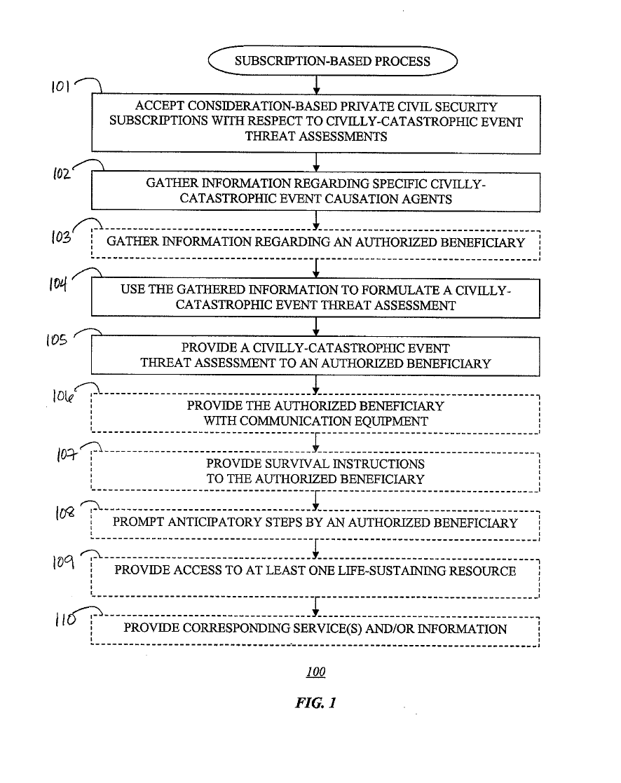

These and other benefits may become clearer upon making a thorough review and study of the following detailed description. Referring now to FIG. 1, these teachings provide a process 100 to facilitate the provisioning of civilly-catastrophic event threat assessments. This process 100 comprises accepting 101 consideration-based private civil security subscriptions from subscribers with respect to providing civilly-catastrophic event threat assessments to an authorized beneficiary. By one approach, these subscriptions may be accepted by, for example, a for-profit business. By another approach, a not-for-profit business (such as a membership-based entity) may be the appropriate entity to offer and accept such subscriptions. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extends past the death of a named subscription beneficiary and further allows for testate and/or intestate transfer to an heir);

rights of access predicated upon a series of periodic payments (as where a subscription provides access rights during, for example, predetermined periods of time on a periodic basis as where a subscriber offers month-by-month payments to gain corresponding month-by-month access rights);

rights of access predicated upon a one-time payment (as may occur when a subscriber makes a single payment to obtain a time based or event-based duration of access rights or, if desired, when a single payment serves to acquire a perpetual right of access that may be retained, transferred, inherited, or the like);

ownership-based rights of access (as may occur when the subscription provides for ownership rights in the at least one life-sustaining resource, when the subscriber is a stockholder of the entity that provides these services, and so forth);

non-transferable rights of access (as may occur when the subscription, by its terms and conditions, prohibits transfer of the right of access to the at least one life-sustaining resource from a first named beneficiary to another);

transferable rights of access (as may occur when the subscription, by its terms and conditions, permits conditional or unconditional transfer of the right of access to the at least one life-sustaining resource from a first named beneficiary to another);

membership-based rights of access (as may occur when the subscription, by its terms and conditions, establishes a membership interest with respect to the accorded right of access such as, for example, a club based membership);

fractionally-based rights of access (as may occur when the subscription, by its terms and conditions, establishes a divided or undivided co-ownership interest by and between multiple subscription beneficiaries with respect to a right to access the at least one life-sustaining resource);

non-ownership-based rights of access (as may occur when the subscription, by its terms and conditions, establishes the aforementioned right of access via, for example, a lease, rental, or borrowing construct); and/or

option-based rights of access.

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

If desired, a plurality of differentiated subscription opportunities can be offered. As but one very simple illustration in this regard, such subscription opportunities can differ from one another at least with respect to cost. This, in turn, provides subscriber choice with respect to selecting a particular subscription that best meets their specific needs and/or budget limitations. Generally, a subscription for the provision of civilly-catastrophic event threat assessments is provided at a predetermined cost as a function of at least one of the following factors: a category of threat assessment information provided (such as naturally-caused disasters or non-naturally-caused disasters, with further subdivisions possible within each category); a level of detail of the threat assessment information provided (such as, for example, geographic area, day, week, or time), a source of threat assessment information (such as, for example, a satellite, a person, or building a relationship with a foreign country was required to gather the information), a number of persons affected by a given potential civilly-catastrophic event (such as, for example, 100 persons affected versus 1,000,000 persons affected), a geographic condition (such as, for example, the information is gathered in a remote mountain range), a seasonal-based condition, a weather condition (such as, for example, the information was gathered during a hurricane), the mode of providing the threat assessment information (such as, for example, by telephone or in-person communication), the frequency of the provision of the threat assessment information (such as, for example, on a daily or weekly basis), or a combination thereof. For example, one subscription can provide daily in-person threat assessment briefings while another less expensive subscription can provide for daily threat assessments via email or telephone. As another example, different subscriptions can be provided that reflect different combinations of categories of alerts. As another example, an authorized beneficiary may desire to receive civilly-catastrophic event threat assessments for several geographic areas, including, for example, areas where relatives of the authorized beneficiary reside. Other possibilities are of course possible.

The process then provides for gathering 102 information regarding specific civilly-catastrophic event causation agents, wherein the specific civilly-catastrophic event includes naturally-caused civilly-catastrophic events as well as non-naturally-caused civilly-catastrophic events.

As used herein, “civilly-catastrophic event” will be understood to refer to an event that substantially and materially disrupts a society's local, regional, and/or national infrastructure and ability to provide in ordinary course at least one life-sustaining resource. Such a civilly-catastrophic event can include both a precipitating event (which may occur over a relatively compressed period of time or which may persist in substantial form over an extended period of time) as well as the resultant aftermath of consequences wherein the precipitating event and/or the resultant aftermath include both the cause of the infrastructure interruption as well as the continuation of that interruption.

A civilly-catastrophic event can be occasioned by any of a wide variety of natural and/or non-naturally caused disasters. Examples of natural disasters that are potentially capable of initiating a civilly-catastrophic event include, but are not limited to, severe weather-related events (such as hurricanes, tsunamis, severe droughts, widespread or unfortunately-targeted tornadoes, severe hail or rain, flooding, and so forth), severe geological events (such as earthquakes, volcanic activity, and so forth), extreme astronomical events (such as Earthly collisions with comets, large asteroids, and so forth, extreme solar flares, and so forth), severe environmental events (such as widespread uncontrolled fire, a rapidly and/or widely dispersed unduly invasive species, and so forth), severe natural shortage of a life-sustaining resource (such as fresh water, food, shelter, and so forth), and global or regional pandemics, to note but a few.

Examples of non-natural disasters capable of initiating a civilly-catastrophic event include both unintentional events as well as intentional acts of war, terrorism, madness, or the like, nuclear-related events (including uncontrolled fission or fusion releases, ionizing radiation exposure, and so forth), the release of deadly or otherwise disruptive biological or chemical agents or creations, a severe concussive event, a severe widespread conflagration event, an event that results in widespread exposure to a mutagenic danger, and so forth.

As used herein, “causation agents” can comprise, for example, naturally-caused civilly-catastrophic event agents such as extreme weather events, extreme geologic events, and so forth. Causation agents can also comprise, if desired, non-naturally-caused civilly-catastrophic event agents. Examples in this regard might include, but are certainly not limited to, prompting or sustaining agents regarding political, religious, ethnic, or other kinds of social unrest.

This information gathered regarding specific civilly-catastrophic event causation agents can comprise, at least in part, information regarding characterizing attributes regarding various civilly-catastrophic events as well as the likelihood of specific civilly-catastrophic events occurring, their causes, their immediate and longer term effects and impact, their relative short term and long term likelihoods of occurring, ways by which such events may be personally anticipated, the extent, severity, and likelihood that an event becomes a civilly-catastrophic event. Gathering 102 the information includes, at least in part, gathering information regarding at least one of the following:

category of threat;

geographic location of threat;

existing weather conditions;

past civilly-catastrophic events of a given nature;

predicted weather conditions;

seasonal-based conditions (as may pertain, for example, to climate, individual and/or crowd behaviors, traffic conditions, supplies and commodities availability, disease vectors and patterns, and so forth);

geographic circumstances (as may pertain, for example, to terrain, body of water, mountain range, desert, and so forth);

a day of the week;

a time of day;

a season;

road conditions;

transport conditions (as may pertain, for example, to whether air, water, or land-based assets are deployable);

a particular person (such as a leader of a country or a terrorist group);

a particular political group;

a particular religious group;

a particular country;

population size;

a number of persons affected by a given potential civilly-catastrophic event;

a level of governmental preparedness for a given civilly-catastrophic event;

location(s) of prepositioned relief supplies;

supply chain locations for at least one life-sustaining resource;

an external threat to rescue personnel;

a residual threat to rescue personnel;

light data (such as, for example, the number of daylight hours dependent on season);

preparedness to combat spread of disease after a given potential civilly-catastrophic event;

and so forth.

By one approach, for example, this gathering 102 of information regarding specific civilly-catastrophic event causation agents can comprise, at least in part, gathering information regarding past, present, and potential civilly-catastrophic events from various resources, including at least of one of public and non-public resources, as desired and available.

By one approach, information may be gathered 102 from a public resource. In this aspect, gathering information from a public resource may comprise arranging to have a relationship with a government entity. For example, the government entity may include a committee, department, agency, commission, bureau, or the like, at the state, local, or federal level. It may be desired that the relationship between the intelligence-gathering entity and the government entity comprises a cooperative, reciprocal relationship where both entities exchange information. It may also be desired that the intelligence-gathering entity and the government entity exchange sensitive information. In this aspect, it may be required that the person or persons gathering the information receive a security clearance from the government in order to facilitate the exchange of sensitive information.

By another approach, information may be gathered 102 from a non-public resource. In this aspect, non-public resources may include, among others, national companies, multinational companies, international organizations (such as the United Nations), private organizations, authorized beneficiary input, monitoring tools, foreign nations, input from professionals, or the like. In one form, the intelligence-gathering entity may arrange to have a relationship with a non-public resource. As one example, the intelligence-gathering entity may arrange to have a relationship with a network of physicians who provide assessments regarding the spread of a disease vector or the dangers associated therewith. As another example, the intelligence-gathering entity may arrange to have a relationship with an international organization or a foreign country.

In another aspect, the non-public resource may comprise at least one monitoring tool, such as one or more surveillance devices, pre-positioned in, near, or above a specific geographic area. The at least one monitoring tool may be used to monitor the conditions of a specific geographic area. The monitoring tools may comprise at least one of: a periscope, a window, a video transmission, a satellite, a photographic transmission, a local sensor, and/or a closed circuit television to note but a few. Such tools may also provide information related to external temperature, air quality, environmental conditions, ionizing radiation, and the scope of damage as created by the occurrence of a civilly-catastrophic causation agent or by a civilly-catastrophic event. In one aspect, such information provided by the monitoring tools may be helpful in determining the extent of a civilly-catastrophic event (i.e., how widespread it is). In another aspect, the monitoring tools may be used to detect a dangerous condition of which an authorized beneficiary may be alerted prior to the authorized beneficiary being exposed to the dangerous condition. In yet another aspect, such information may also be helpful in determining when a danger associated with a civilly-catastrophic event has dissipated such that the authorized beneficiary may safety resume normal activities.

In this aspect, for example, the at least one monitoring tool may be positioned about a metropolitan area, tourist attraction, mass transit facility or vehicle (such as ferries, trains, buses, airplanes, or the like), national monument, airport, high-rise building or skyscraper, bridge, stadium, school, financial center, cruise ship, or other location where large numbers of people are likely to congregate.

In another aspect, the at least one monitoring tool may comprise a local sensor configured and arranged to detect at least one of the following: ionizing radiation; a chemical agent; a biological agent; a seismic event; a weather condition; a sonic event; a concussive event; a thermal event; a civil disturbance; or a biological disturbance.

In another aspect, the authorized beneficiary may assist in the information gathering step of the process. As but one example, at least one monitoring tool may be positioned at the home or office, in the vehicles, or on the person of an authorized beneficiary. In this aspect, the authorized beneficiary may play an important role in transmitting information relating to the monitoring tool to the intelligence gathering entity, particularly in instances when monitoring tools are positioned at the home or on the person of a plurality of authorized beneficiaries. Other examples no doubt exist.

If desired, the process 100 may optionally accommodate gathering 103 information regarding an authorized beneficiary. Such information could include the authorized beneficiary's physical condition (including age, physical or mental disability, or special medical needs (such as chronic prescriptions, allergies, or asthma)), identification information, contact information (such as at home and at work), political and/or religious affiliation, or racial and/or cultural standing or affinity. Such information could also include details regarding the authorized beneficiary's schedule, such as when the authorized beneficiary is likely to be away from home on a scheduled basis (for example, at work, at school, at a health club, at a volunteer activity, or the like). The information gathered 102 from the authorized beneficiary could also include preferred destination information and evacuation plan information. The information regarding the authorized beneficiary might serve, for example, to aid in facilitating delivery of the threat assessment to the authorized beneficiary on an expedited basis when a civilly-catastrophic event has occurred or is likely to occur, and would also assist in determining the most efficient way to provide additional services to the authorized beneficiary, such as a rescue service.

This process 100 then provides for using 104 the gathered information, at least in part, to formulate a civilly-catastrophic event threat assessment. In one aspect, events of likely greater civil magnitude, for example, perhaps more likely garner a projection mention notwithstanding a present relatively low likelihood of occurring. In another aspect, events likely to occur within a predetermined period of time, for example, perhaps more likely garner a projection mention notwithstanding a present relatively low likelihood of occurring.

By one approach, such gathered information may be used 104 to formulate civilly-catastrophic event threat assessments that are local to a particular limited geographic area (such as a given city (or portion of a city), county, state, or the like).

By another approach, such gathered information may be used 104 to formulate civilly-catastrophic event threat assessments that are particular to an authorized beneficiary who comprises a particular cultural (such as, but not limited to, political, religious, ethnic, tribal, racial, or the like) affiliation, sexual affiliation, economic status, group affiliation (such as, but not limited to, clubs, unions, schools, societies, or the like), corporate affiliation, or the like. In this aspect, the gathered information regarding the authorized beneficiary may be used to formulate a personalized civilly-catastrophic event threat assessment.

By these approaches, the civilly-catastrophic event threat assessments are formulated such that an authorized beneficiary would tend to receive threat assessment information of particular personal relevance while not necessarily being similarly exposed to less relevant information. Other bases and criteria for targeting particular threat assessment information for particular authorized beneficiaries may of course be considered and applied in accordance with the needs, requirements, and/or options presented in a given application setting.

This process 100 further includes providing 105 the authorized beneficiary of one of the consideration-based private civil security subscriptions with the civilly-catastrophic event threat assessment related to any of a plurality of categorically different civilly-catastrophic events. The threat assessment provided to an authorized beneficiary may comprise at least one of the following:

a location of a potential civilly-catastrophic event;

a location of an already-having-occurred civilly-catastrophic event;

a likely magnitude of civil impact of a given potential civilly-catastrophic event;

a likelihood of a given civilly-catastrophic event to trigger another civilly-catastrophic event;

a likelihood of a civilly-catastrophic event occurring;

a geographic scope of a potential civilly-catastrophic event;

a likely timing of a potential civilly-catastrophic event;

a likely scope of societal disruption of a potential civilly-catastrophic event;

a likelihood that a potential civilly-catastrophic event will disrupt a supply chain for at least one life-sustaining resource;

a likely duration of a given potential civilly-catastrophic event;

a likely long term effect of a given potential civilly-catastrophic event;

a likely short term effect of a given potential civilly-catastrophic event;

a likelihood of a given potential civilly-catastrophic event to affect habitability of a given geographic area;

predicted weather conditions.

There are numerous and various ways by which such threat assessments can be provided. For example, such threat assessments can be provided 105 via delivery of hardcopy to the authorized beneficiary, via facsimile transmission, via email, via voicemail, via text messaging, via telephony, via a radio broadcast (such as a public, private, satellite, or two-way radio broadcast), via a personal data assistant, via a public or private television broadcast, via a portable electronic wireless receiver, via a point-to-multipoint telecommunication, via a non-verbal symbol, via smoke signal, via a light shined on a surface (such as a building) or the sky, via an electronic or digital sign, via a siren or siren-like announcement, via a loudspeaker, via a flag or banner, via an in-person communication, via a secure password-protected website, and so forth. In another aspect, the threat assessments can be provided by using a variety of different languages or by using pictorial or graphic symbols (such as, but not limited to, universal symbols). The mode by which the threat assessment information is provided may be as desired by the authorized beneficiary. As one example, the authorized beneficiary may desire daily or weekly in-person briefings. As another example, the authorized beneficiary may desire daily or weekly emails or updates provided through a password-protected secure website.

Such threat assessment information can be provided 105 on a relatively unsynchronized and/or irregular manner or, if desired, can be provided on a relatively frequent and periodic basis. In another aspect, such threat assessment information can be provided on a relatively frequent and periodic basis notwithstanding a present relatively low likelihood of a civilly-catastrophic event occurring. For example, such threat assessment information can be provided on a daily or weekly basis, depending on the desires of the authorized beneficiary. Alternatively, the authorized beneficiary may desire to receive threat assessments only a needs-to-know basis, such as when a civilly-catastrophic event is predicted or when there is a heightened risk of a civilly-catastrophic event occurring.

In another aspect, the mode of providing the threat assessment information may depend on the severity or predicted timing of the civilly-catastrophic event. The mode by which a threat assessment is provided can vary dramatically depending on the category of civilly-catastrophic event being addressed. In some cases, a very rapid communication may be essential (such as by telephone) while in other cases a more sedate, deliberate approach may be viable. For example, a widespread uncontrolled fire requires providing a threat assessment by a more immediate mode of communication (such as by telephone) than a forecast drought.

In an optional aspect, the process 100 may further comprise providing 106 the authorized beneficiary with communication equipment. In one aspect, the communication equipment is operable to receive civilly-catastrophic event threat assessment information. In another aspect, it may be desired that the communication equipment be used to facilitate two-way communication between the authorized beneficiary and at least one of: other authorized beneficiaries, a private civil-defense service, a public emergency service, and a public member. In this aspect, it may be desired that the communication equipment be used to obtain information about the authorized beneficiary, including the authorized beneficiary's condition and location, among others.

This process 100 may optionally further include, if desired, providing 107 the authorized beneficiary of one of the consideration-based private civil security subscriptions with survival instructions. The survival instructions may include instructions on general actions that an authorized beneficiary can take to better prepare to at least survive a given civilly-catastrophic event, such as the identification of particular supplies that are useful to obtain prior to a civilly-catastrophic event, identification of particular suppliers of recommended survival supplies, identification of particular precautions to take to better facilitate surviving a civilly-catastrophic event, identification of recommended actions to take to better facilitate surviving a civilly-catastrophic event, identification of recommended usage of survival-related supplies when surviving a civilly-catastrophic event, identification of recommended evacuation routes, or the like.

Those skilled in the art will understand and appreciate that such survival instructions may be provided 107 during a time of immediate need but may also be provided prior to the need actually arising. For example, such information can be provided from time to time in order to keep authorized beneficiaries apprised of what evacuation route they could or should use in the event a civilly-catastrophic event (whether forecast or not) occurs. This can be particularly important, for example, in event scenarios where modes of communication are partially or fully disabled during the actual time of need.

As another example, in one case a threat assessment may represent a very short term event (such as hours or only one day or so) while in other cases the threat assessment may represent a longer term event (such as many days or even weeks or months). Such differences, in turn, can lead to significant differences with respect to the recommended survival instructions that may be provided 107 to an authorized beneficiary regarding specific actions that may or should be reasonably taken under such circumstances.

The survival instructions may also comprise information to keep an authorized beneficiary apprised of the safest modes and routes of travel. For example, different airports have varying levels of security equipment. An authorized beneficiary may desire to direct travel plans through airports having the highest level of security equipment. An authorized beneficiary may also wish to direct travel plans through cities and/or countries having a stable government and not currently experiencing civil upheaval or spread of a life-threatening disease vector.

Viewed generally, for example, such survival instructions could also serve to facilitate an ability of the authorized beneficiaries to better comply with the terms and conditions of their subscriptions. To illustrate, such survival instructions could comprise information regarding shelter facilities, recommended behaviors, the contents, storage, and usage of survival-related supplies as are provided to the authorized beneficiaries pursuant to their subscriptions, and so forth. Such survival instructions could also comprise information related to locating, boarding, and traveling in pre-arranged subscription-based transport and/or how to cooperate with rescue personnel to aid with effecting one's own rescue and/or extraction.

The process 100 may optionally further comprise prompting 108 particular anticipatory steps by the authorized beneficiary such as, among others, one of the following: the stockpiling of particular commodities (such as, but not limited to, necessities (including water and nourishment)); the pre-placement of particular evacuation supplies; an adjustment with respect to ordinary daily behaviors; training to better facilitate surviving a civilly-catastrophic event; evacuation; locating, boarding, and traveling in pre-arranged subscription based transport; usage of survival-related supplies; contacting and locating family members of the authorized beneficiary; formulating an emergency shelter using substantially only commonly available household items. Such information generally comprises instructions regarding specific actions that an authorized beneficiary can take in a relatively immediate timeframe in order to better protect him or herself from a given civilly-catastrophic event.

The process 100 may optionally further encompass providing 109 access to at least one life-sustaining resource by the entity providing the civilly-catastrophic event threat assessments. For example, the at least one life-sustaining resource may pertain to hydration and nutritional consumables (such as, but not limited to, water and food), clothing, private civil defense shelter, private civil defense transport services, environmentally-borne threat abatement (including, for example, personally worn items such as breathing masks, special clothing, and so forth), medical facilities, compressed or stored air, private civil defense rescue services, and/or providing of privately developed customized instructions regarding appropriate survival actions to take in response to a civilly-catastrophic event. Numerous examples of such services are set forth in the various patent applications noted above.

As yet another example, these teachings will further optionally support the provision 110 of other corresponding service(s) and/or information. In one aspect, this can comprise providing a communications service via, for example, a voicemail host, a bulletin board host, website, radio station, television station, billboard, electronic sign board, satellite signal, and/or an email host. In such a case, if desired, the corresponding information can include information regarding how to access and utilize such hosts such that the authorized beneficiary can utilize the host to re-establish contact with predetermined other persons upon the occurrence of a civilly-catastrophic event. As another example, this can include providing threat assessment information to the at least one person (via any communication vehicle of choice including but not limited to email, a hardcopy or virtual newsletter, a website, and so forth).

So configured, these teachings provide a powerful, economical, highly scalable, and readily leveraged mechanism by which a given authorized beneficiary or group of authorized beneficiaries can be provided with a well-informed yet personally customized civilly-catastrophic event threat assessments.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

What is claimed is:

1. A method comprising:
accepting consideration-based private civil security subscriptions from subscribers with respect to providing the civilly-catastrophic event threat assessments to an authorized beneficiary;
gathering information regarding specific civilly-catastrophic event causation agents;
using the information, at least in part, to formulate a civilly-catastrophic event threat assessment;
providing to the authorized beneficiary of one of the consideration-based private civil security subscriptions the civilly-catastrophic event threat assessment related to any of a plurality of categorically different civilly-catastrophic events having at least one of:
occurred; and
potentially occurring;
such that an authorized beneficiary of a consideration-based private civil security subscription is able to reliably and predictably receive specific information regarding potential civilly-catastrophic events.

2. The method of claim 1 wherein an entity providing the civilly-catastrophic event threat assessment also provides civilly-catastrophic event-based access to at least one life-sustaining resource.

3. The method of claim 2 wherein the at least one life-sustaining resource comprises at least one of:
hydration;
nourishment;
a life-sustaining shelter;
transport away from an area of substantially sudden civil upheaval;
environmentally-borne threat abatement;
compressed or stored air; and
a rescue service to come to an authorized beneficiary as corresponds to one of the subscriptions and move the authorized beneficiary away from a location of substantially sudden civil upheaval.

4. The method of claim 1 wherein gathering information regarding specific civilly-catastrophic event causation agents comprises gathering information from at least one of:
a public resource;
a non-public resource.

5. The method of claim 4 wherein the non-public resource comprises an authorized beneficiary input.

6. The method of claim 4 wherein the non-public resource comprises at least one monitoring instrument pre-positioned in a specific geographic area.

7. The method of claim 6 wherein the at least one monitoring instrument is a local sensor configured and arranged to detect at least one of the following:
ionizing radiation;
a chemical agent;
a biological agent;
a seismic event;
a weather condition;
a sonic event;
a thermal event;
a civil disturbance;
a biological disturbance.

8. The method of claim 1 wherein using the information, at least in part, to formulate a civilly-catastrophic event threat assessment comprises using the information to formulate a civilly-catastrophic event threat assessment that is specifically targeted for individuals who comprise at least one of:
individuals within a particular geographic area;
individuals of a particular cultural affiliation;
individuals of a particular religious affiliation;
individuals of a particular economic affiliation;
individuals of a particular ethnic affiliation;
individuals of a particular tribal affiliation;
individuals of a particular group affiliation;
individuals of a particular corporate affiliation.

9. The method of claim 1 wherein:
using the information, at least in part, to formulate a civilly-catastrophic event threat assessment comprises using the information to formulate a civilly-catastrophic event threat assessment that is local to a particular limited geographic area;
providing to an authorized beneficiary of one of the consideration-based private civil security subscriptions the civilly-catastrophic event threat assessment comprises providing the civilly-catastrophic event threat assessment that is local to a particular limited geographic area in a limited manner that tends to include authorized beneficiaries located within the particular limited geographic area and exclude authorized beneficiaries located outside the particular limited geographic area.

10. The method of claim 1 wherein gathering the information, comprises, at least in part, gathering information regarding at least one of the following:
a category of threat;
geographic location of threat;
existing weather conditions;
past civilly-catastrophic events of a given nature;
predicted weather conditions;
seasonal-based conditions;
geographic circumstances;
terrain;
a time of day;
a day of the week;
season;
road conditions;
transport conditions;
a particular person;
a particular political group;
a particular religious group;
a particular country;
population size;
a number of persons affected by a given potential civilly-catastrophic event;
a level of governmental preparedness for a given potential civilly-catastrophic event;
location(s) of pre-positioned relief supplies;
supply chain locations for at least one life-sustaining resource;
external threats to rescue personnel;
residual threats to rescue personnel;
light data;
preparedness to combat spread of disease after a given potential civilly-catastrophic event.

11. The method of claim 1 wherein accepting consideration-based private civil security subscriptions from subscribers comprises accepting the consideration-based private civil security subscriptions at a for-profit business.

12. The method of claim 1 wherein the civilly-catastrophic event comprises an event that substantially disrupts society's infrastructure and ability to provide at least one life-sustaining resource.

13. The method of claim 1 wherein the civilly-catastrophic event is one that is likely to persist in substantial form for more than a predetermined period of time.

14. The method of claim 1 wherein the civilly-catastrophic event comprises at least one of:
a natural disaster;
a non-naturally-caused disaster.

15. The method of claim 14 wherein the natural disaster comprises at least one of:
a severe weather event;
a severe geological event;
a severe geophysical event;
a severe astronomical event;
a severe disease-based event;
a severe natural shortage of a life-sustaining resource.

16. The method of claim 14 wherein the non-naturally-caused disaster comprises at least one of:
an intentional act of aggression;
an unintentional act of aggression;
an unintended event that results in public dispersal of a severe environmentally borne danger;
a release of nuclear radiation event;
a release of at least one dangerous biological agent event;
a release of at least one dangerous chemical agent event;
a severe widespread conflagration event;
an act of war event;
an act of terrorism event;
an act of madness;
a severe concussive event;
an event that results in widespread exposure to a mutagenic danger.

17. The method of claim 1 wherein the civilly-catastrophic event threat assessment provided to an authorized beneficiary comprises at least one of:
a location of a potential civilly-catastrophic event;
a location of an already having occurred civilly-catastrophic event;
a likely magnitude of civil impact of a given potential civilly-catastrophic event;
a likelihood of a given civilly-catastrophic event to trigger another civilly-catastrophic event;
a likelihood of a civilly-catastrophic event occurring;
a geographic scope of a potential civilly-catastrophic event;
a likely timing of a potential civilly-catastrophic event;
a likely scope of societal disruption due to a potential civilly-catastrophic event;
a likelihood that a potential civilly-catastrophic event will disrupt a supply chain for at least one life-sustaining resource;
a likely duration of a given potential civilly-catastrophic event;
a likely long-term effect of a given potential civilly-catastrophic event;
a likely short-term effect of a given potential civilly-catastrophic event;
a likelihood of a given potential civilly-catastrophic event to affect habitability of a given geographic area;
predicted weather conditions.

18. The method of claim 1 further comprising providing survival instructions to the authorized beneficiary.

19. The method of claim 15 wherein the survival instructions comprise at least one of:
identification of particular supplies that are useful to obtain prior to a civilly-catastrophic event;
identification of particular suppliers of recommended survival supplies;
identification of particular precautions to take to better facilitate surviving a civilly-catastrophic event;
identification of recommended actions to take to better facilitate surviving a civilly-catastrophic event;
identification of recommended usage of survival-related supplies when surviving a civilly-catastrophic event;
identification of recommended evacuation routes.

20. The method of claim 1 further comprising prompting particular anticipatory steps by the authorized beneficiary.

21. The method of claim 20 wherein the particular anticipatory steps by the authorized beneficiary comprise at least one of:
training to better facilitate surviving a civilly-catastrophic event;
stockpiling particular commodities;
pre-placement of particular evacuation supplies;
an adjustment with respect to ordinary daily behaviors;
evacuation;
locating, boarding, and traveling in pre-arranged subscription-based transport;
usage of survival-related supplies;
contacting and locating family members of authorized beneficiaries;
formulating an emergency shelter using substantially only commonly available household items.

22. The method of claim 20 wherein prompting particular anticipatory steps by the authorized beneficiary comprises providing instructions regarding specific actions that an authorized beneficiary can take in a relatively immediate timeframe in order to better protect himself or herself from a given civilly-catastrophic event.

23. The method of claim 1 wherein the civilly-catastrophic event threat assessment is provided via at least one of the following modes of communication:
email;
facsimile;
voicemail;
text messaging;
a secure website;
telephony;
a public radio broadcast;
a private radio broadcast;
a two-way radio broadcast;
a satellite radio broadcast;
a personal data assistant;
a public television broadcast;
a private television broadcast;
a portable electronic wireless receiver;
a point-to-multipoint telecommunication;
a briefing on a predetermined basis;
a non-verbal symbol;
printed media;
an in-person communication;
a smoke signal;
a siren;
a loudspeaker;
a flag;
a banner;
an electronic sign;
a digital sign;
a light.

24. The method of claim 1 further comprising providing the authorized beneficiary with communication equipment operable to receive civilly-catastrophic event threat assessment information.

25. The method of claim 24 further comprising using the communication equipment to obtain information about the authorized beneficiary.

26. The method of claim 24 further comprising using the communication equipment to facilitate two-way communication between the authorized beneficiary and at least one of:
other authorized beneficiaries;
a private civil-defense service;
a public emergency service;
a public member.

27. The method of claim 1 further comprising gathering information about the authorized beneficiary, wherein the gathered information comprises at least one of:
identification information;
contact information;
location information;
environmental condition information;
health condition information;
preferred destination information;
evacuation plan information.

28. The method of claim 1 wherein the civilly-catastrophic event threat assessment is provided at a predetermined cost as a function of at least one of the following:
a category of threat assessment information provided;
a source of threat assessment information;
a number of persons affected by a given potential civilly-catastrophic event;
a geographic condition;
a seasonal-based condition;
a level of detail of the threat assessment provided;
a mode of communicating the threat assessment;
a day of week;
a time of day;
weather conditions;
light data;
a frequency of the provision of the threat assessment.

29. The method of claim 1 wherein providing the civilly-catastrophic event threat assessment related to any of a plurality of categorically different civilly-catastrophic events comprises providing the civilly-catastrophic event threat assessment on a relatively frequent and periodic basis.

30. The method of claim 29 wherein providing the civilly-catastrophic event threat assessment on a relatively frequent and periodic basis comprises transmitting information regarding civilly-catastrophic event threat assessments on a relatively frequent and periodic basis notwithstanding a present relatively low likelihood of a civilly-catastrophic event occurring.

31. The method of claim 1 wherein the subscriptions comprise at least one of:
time-limited rights of access;
event-limited rights of access;
inheritable rights of access;
rights of access predicated upon a series of periodic payments;
rights of access predicated upon a one-time payment;
ownership based rights of access;
non-transferable rights of access;
transferable rights of access;
membership-based rights of access;
fractionally-based rights of access;
non-ownership-based rights of access;
option-based rights of access.
